(19) United States
(12) Patent Application Publication
(10) Pub. No.: US 2021/0202078 A1
(43) Pub. Date: Jul. 1, 2021

(54) Patient-Observer Monitoring

(71) Applicant: CERNER INNOVATION, INC., KANSAS CITY, KS (US)

(72) Inventor: Greg Ford, Shawnee, KS (US)

(21) Appl. No.: 17/003,511

(22) Filed: Aug. 26, 2020

Related U.S. Application Data
(60) Provisional application No. 62/953,776, filed on Dec. 26, 2019.

(51) Int. Cl. G16H 40/20; G16H 40/67; G16H 50/30; G16H 50/70; G16H 10/60; G06K 9/00; A61B 5/16; A61B 5/00; A61B 5/11; A61B 5/0205; A61B 5/0476

(52) U.S. Cl. CPC G16H 40/20; A61B 5/024; G16H 50/30; G16H 50/70; G16H 10/60; G06K 9/00342; G06K 9/00302; A61B 5/162; A61B 5/746; A61B 5/1116; A61B 5/163; A61B 5/0205; A61B 5/0476; A61B 5/1117; A61B 5/748; G16H 40/67

(57) ABSTRACT

Methods, systems, and computer-readable media for patient-observer monitoring are provided herein. A method creates a reference model based on interaction data of previous patient-observers. The method determines an optimal value for alertness based on the reference model. The method detects interaction data for a particular patient-observer. The method calculates an alertness value for the particular patient-observer. The method generates a warning when the calculated alertness value does not satisfy the optimal value.

### CROSS REFERENCE TO RELATED APPLICATION

This patent application is a nonprovisional application that claims the benefit of and priority to U.S. Provisional App. No. 62/953,776, filed on Dec. 26, 2019, the entirety of which is incorporated herein by reference.

### BACKGROUND

Patients are often monitored within a healthcare facility. One reason to monitor patients is to redirect risky behavior or address a patient's immediate needs. Monitoring patients allows for observing a change in a patient's condition quickly and accurately. For example, monitoring patients could prevent a patient from falling off of a bed. Falls are a leading cause of death among people over the age of 65 years, and 10% of the fatal falls for patients over 65 years of age occur in a hospital setting. As another example, monitoring patients allows for the detection of stroke symptoms. The occurrence of a stroke calls for prompt attention.

Some problems surrounding monitoring patients to prevent falls, strokes, or other risks involve the actual monitoring of the patient. For example, equipment used to monitor the patient may not be utilized properly. A headset may not be enabled to detect audible cues, or the headset may not be utilized if shared with more than one person. Additionally, some who monitor patients are working long shifts, monitoring more than ten patients at one time, or both.

### SUMMARY

This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

Technologies described herein generally relate to devices, methods, systems, or instructions embodied on one or more non-transitory computer-readable media for monitoring a patient-observer. In an aspect, the non-transitory computer-readable media may perform a method. The method may include creating a reference model based on interaction data of previous patient-observers; determining an optimal value for alertness based on the reference model; detecting, using at least one sensor, interaction data for a particular patient-observer; calculating an alertness value for the particular patient-observer based on the reference model and the interaction data for the particular patient-observer; and generating a warning when the calculated alertness value of the particular patient-observer does not satisfy the optimal value.

Another aspect may include a computerized method for optimizing patient observation. The computerized method may comprise determining a preferred interaction rate for a particular patient-observer based on interaction data of previous patient-observers, the interaction data maintained in a log file within a database; detecting interaction data for the particular patient-observer; determining an interaction rate for the particular patient-observer based on the detected interaction data; and generating a warning when the interaction rate does not satisfy the preferred interaction rate.

Yet another aspect may include a system for monitoring a patient-observer. The system may comprise a database comprising interaction data within a log file, a sensor for detecting real-time interaction data of a particular patient-observer, and one or more processors. The one or more processors may be configured to create a reference model from the interaction data within the log file of the database; detect, using the sensor, real-time interaction data for the particular patient-observer; update the reference model using the detected real-time interaction data; determine a threshold value for alertness from the reference model; calculate an alertness value for the particular patient-observer; and generate a warning when the alertness value for the particular patient-observer fails to satisfy the threshold value of the reference model.

Additional objects, advantages, and novel features of the technology are described below in the Detailed Description.

### BRIEF DESCRIPTION OF THE DRAWINGS

This disclosure references the attached drawing figures, wherein:

FIG. 1 is a simplified schematic view of an exemplary computing environment, in accordance with aspects of this disclosure.

FIG. 2 is a view of an exemplary central monitoring system, in accordance with aspects of this disclosure.

FIG. 3 is a view of an exemplary observation environment, in accordance with aspects of this disclosure.

FIG. 4 is a simplified schematic view of an exemplary system for monitoring a patient-observer, in accordance with aspects of this disclosure.

FIG. 5 is a view of an exemplary sensory environment for the patient-observer, in accordance with aspects of this disclosure.

FIG. 6A is a view of an exemplary log file within a database, in accordance with aspects of this disclosure.

FIG. 6B is a view of an exemplary logged interaction time within the log file, in accordance with aspects of this disclosure.

FIGS. 7-10 are exemplary flow diagrams for monitoring the patient-observer, in accordance with aspects of this disclosure.

FIG. 11 is a view of an exemplary alert, in accordance with aspects of this disclosure.

### DETAILED DESCRIPTION

The subject matter of the present technology is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

As one skilled in the art will appreciate, embodiments of the present technology may be embodied as, among other things: a method, system, or set of instructions embodied on one or more non-transitory computer-readable media, which is described herein. Accordingly, the embodiments may take the form of a hardware embodiment, a software embodiment, or an embodiment combining software and hardware. In one embodiment, the present technology takes the form of a computer-program product that includes computer-usable instructions embodied on one or more non-transitory computer-readable media.

At a high level, this disclosure describes, among other things, technologies for monitoring a patient-observer. Employing patient-observer monitoring in clinical care environments assists in the improvement of quality of patient care. For example, employing patient-observer monitoring can enhance and hasten the detection of patients falling, having strokes, or other risks to a patient. Early detection of a risk often prevents further damage from occurring. In aspects, patient-observer monitoring may be employed in conjunction with prior-established clinical processes to enhance the quality of patient care.

Previous relevant technologies have not provided for the optimization of a patient-observer who monitors patients. Rather, previous relevant technologies have provided for various ways to monitor patients. A lack of alertness could result in a lack of optimal monitoring. For instance, someone who is lacking alertness could miss an audible, visual, or haptic cue that requires attention. Thus, it is advantageous to improve the monitoring of patients. Further, previous relevant technologies have not provided methods, systems, and non-transitory computer-readable media providing a method for determining an optimal value for alertness based on a created reference model, as described herein. Additionally, previous relevant technologies have not provided methods, systems, and non-transitory computer-readable media providing a method for calculating an alertness value for a patient-observer based on the reference model and interaction data, and generating a warning when the calculated alertness value does not satisfy the optimal value for alertness.

Various indications of alertness include, but are not limited to, heart rate, respiration rate, blood oxygen level, facial expressions, head posture, slouching, brain activity (e.g. voltage), voice alterations, eye metrics, and autonomic nervous system activity. Fatigue may be mental or physical. Mental fatigue includes an impairment in judgment, reaction time, and situational awareness. Physical fatigue includes the capacity to perform an amount and an intensity of physical activity for a period of time. Mental fatigue can impact a person physically. People sometimes experience insomnia-related daytime fatigue and excessive daytime sleepiness. Those with daytime fatigue are very tired but usually do not fall asleep, whereas those with excessive daytime sleepiness feel drowsy during the day and typically fall asleep during the day when bored or in a sedentary situation. Alertness may include, but is not limited to, mental fatigue, physical fatigue, sleepiness, a degree of arousal on a sleep-wake axis, a level of cognitive performance, behavior awareness, drowsiness, vigilance, sustained attention for a period of time, and tonic alertness. Various methods for detecting and evaluating alertness are discussed below in more detail. Additionally, as new sensors and improvements to existing sensors are developed, they may be incorporated into methods and systems disclosed herein.

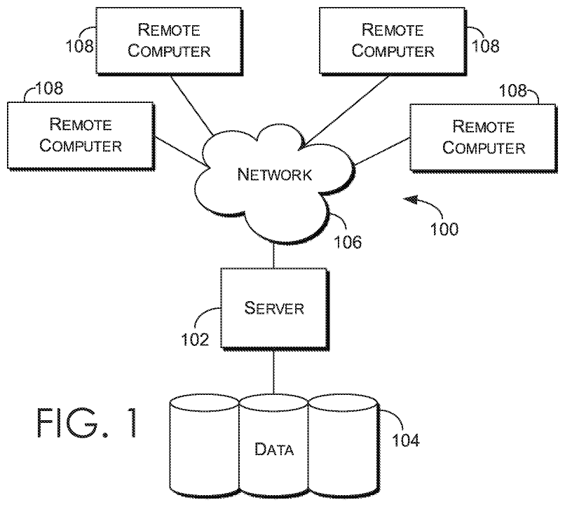

Beginning with FIG. 1, an exemplary computing environment suitable for use in implementing embodiments of the present technology is shown. FIG. 1 is an exemplary computing environment (e.g., health-information computing-system environment) with which embodiments of the present technology may be implemented. The computing environment is illustrated and designated generally as reference numeral 100. The computing environment 100 is merely an example of one suitable computing environment and is not intended to suggest any limitation as to the scope of use or functionality of the present technology. Neither should the computing environment 100 be interpreted as having any dependency or requirement relating to any single component or combination of components illustrated therein. It will be appreciated by those having ordinary skill in the art that the connections illustrated in FIG. 1 are also exemplary as other methods, hardware, software, and devices for establishing a communications link between the components, devices, systems, and entities, as shown in FIG. 1, may be utilized in the implementation of the present technology. Although the connections are depicted using one or more solid lines, it will be understood by those having ordinary skill in the art that the exemplary connections of FIG. 1 may be hardwired or wireless, and may use intermediary components that have been omitted or not included in FIG. 1 for simplicity's sake. As such, the absence of components from FIG. 1 should not be interpreted as limiting the present technology to exclude additional components and combination(s) of components. Moreover, though devices and components are represented in FIG. 1 as singular devices and components, it will be appreciated that some embodiments may include a plurality of the devices and components such that FIG. 1 should not be considered as limiting the number of a device or component.

The present technology might be operational with numerous other special-purpose computing system environments or configurations. Examples of well-known computing systems, environments, and/or configurations that might be suitable for use with the present technology include personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

The present technology may be operational and/or implemented across computing system environments such as a distributed or wireless “cloud” system. Cloud-based computing systems include a model of networked enterprise storage where data is stored in virtualized storage pools. The cloud-based networked enterprise storage may be public, private, or hosted by a third party, in embodiments. In some embodiments, computer programs or software (e.g., applications) are stored in the cloud and executed in the cloud. Generally, computing devices may access the cloud over a wireless network and any information stored in the cloud or computer programs run from the cloud. Accordingly, a cloud-based computing system may be distributed across multiple physical locations.

The present technology might be described in the context of computer-executable instructions, such as program modules, being executed by a computer. Exemplary program modules comprise routines, programs, objects, components, and data structures that perform particular tasks or implement particular abstract data types. The present technology might be practiced in distributed computing environments where tasks are performed by remote processing devices that are linked through a communications network. In a distributed computing environment, program modules might be located in association with local and/or remote computer storage media (e.g., memory storage devices).

With continued reference to FIG. 1, the computing environment 100 comprises a computing device in the form of a control server 102. Exemplary components of the control server 102 comprise a processing unit, internal system memory, and a suitable system bus for coupling various system components, including database 104, with the control server 102. The system bus might be any of several types of bus structures, including a memory bus or memory controller, a peripheral bus, and a local bus, using any of a variety of bus architectures. Exemplary architectures comprise Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The control server 102 typically includes therein, or has access to, a variety of non-transitory computer-readable media. Computer-readable media can be any available media that might be accessed by control server 102, and includes volatile and nonvolatile media, as well as, removable and nonremovable media. By way of example, and not limitation, computer-readable media may comprise computer storage media and communication media. Computer storage media includes volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by control server 102. Computer-readable media does not include signals per se.

Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

The control server 102 might operate in a computer network 106 using logical connections to one or more remote computers 108. Remote computers 108 might be located at a variety of locations including operating systems, device drivers and the like. The remote computers might also be physically located in traditional and nontraditional clinical environments so that the entire medical community might be capable of integration on the network. The remote computers might be personal computers, servers, routers, network PCs, peer devices, other common network nodes, or the like and might comprise some or all of the elements described above in relation to the control server. The devices can be personal digital assistants or other like devices. Further, remote computers may be located in a variety of locations including in a medical or research environment, including clinical laboratories (e.g., molecular diagnostic laboratories), hospitals and other individual settings, veterinary environments, ambulatory settings, medical billing and financial offices, hospital administration settings, home medical environments, and clinicians' offices. Medical providers may comprise a treating physician or physicians; specialists such as surgeons, radiologists, cardiologists, and oncologists; emergency medical technicians; physicians' assistants; nurse practitioners; nurses; nurses' aides; pharmacists; dieticians; microbiologists; laboratory experts; laboratory technologists; genetic counselors; researchers; veterinarians; students; and the like. The remote computers 108 might also be physically located in nontraditional clinical environments so that the entire medical community might be capable of integration on the network. The remote computers 108 might be personal computers, servers, routers, network PCs, peer devices, other common network nodes, or the like and might comprise some or all of the elements described above in relation to the control server 102. The devices can be personal digital assistants or other like devices.

Computer networks 106 comprise local area networks (LANs) and/or wide area networks (WANs). Such networking environments are commonplace in offices, enterprise-wide computer networks, intranets, and the Internet. When utilized in a WAN networking environment, the control server 102 might comprise a modem or other means for establishing communications over the WAN, such as the Internet. In a networking environment, program modules or portions thereof might be stored in association with the control server 102, the database 104, or any of the remote computers 108. For example, various application programs may reside on the memory associated with any one or more of the remote computers 108. It will be appreciated by those of ordinary skill in the art that the network connections shown are exemplary and other means of establishing a communications link between the computers (e.g., control server 102 and remote computers 108) might be utilized.

In operation, an organization might enter commands and information into the control server 102 or convey the commands and information to the control server 102 via one or more of the remote computers 108 through input devices, such as a keyboard, a microphone (e.g., voice inputs), a touchscreen, a pointing device (commonly referred to as a mouse), a trackball, or a touch pad. Other input devices comprise satellite dishes, scanners, or the like. Commands and information might also be sent directly from a remote medical device to the control server 102. In addition to a monitor, the control server 102 and/or remote computers 108 might comprise other peripheral output devices, such as speakers and a printer.

Although many other internal components of the control server 102 and the remote computers 108 are not shown, such components and their interconnection are well known. Accordingly, additional details concerning the internal construction of the control server 102 and the remote computers 108 are not further disclosed herein.

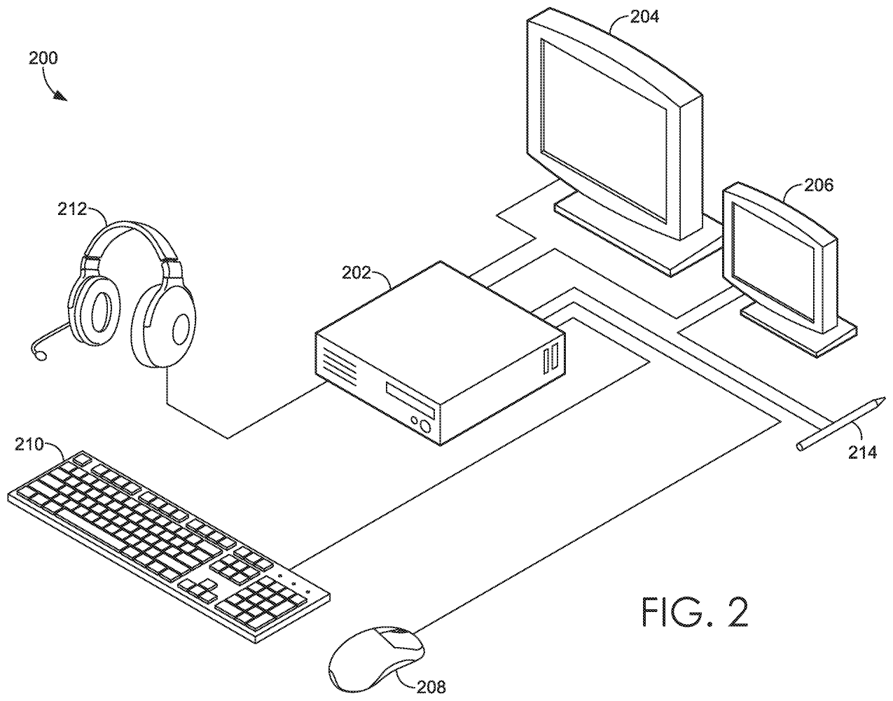

Turning to FIG. 2, exemplary central monitoring system 200 comprises a remote computer 202, a primary monitor 204, a user interface 206, a mouse 208, a keyboard 210, a headset 212, and a drawing tool 214. In one aspect, the exemplary central monitoring system 200 is central to the patient-observer. In another aspect, the exemplary central monitoring system 200 operates as a system configuring and analyzing data from the patient-observer and patients that the patient-observer is monitoring. For example, the exemplary central monitoring system 200 may recognize that a patient the patient-observer is monitoring has moved in a particular way and the patient-observer has not interacted with the exemplary central monitoring system 200 for a specified amount of time (e.g. the patient-observer has not interacted with the mouse 208 for over forty seconds or the drawing tool for over two minutes).

The exemplary central monitoring system 200 may be remotely located at a physical location with a data connection (e.g. USB, TCP/IP, etc.) to devices for observing a patient in real-time. The exemplary central monitoring system 200 may be on the same floor as the patient, on a different floor than the patient, in the same building as the patient, or in a different building than the patient. If the exemplary central monitoring system 200 is monitoring more than one patient, the patients may be located in different rooms, floors, or buildings from one another. The exemplary central monitoring system 200 may be in a single location or may be distributed amongst multiple locations.

The remote computer 202 may comprise a database in communication with a processor. The database may store information received from the primary monitor 204, the user interface 206, the mouse 208, the keyboard 210, the headset 212, and the drawing tool 214. For example, the database may store information pertaining to how often the patient-observer clicks the mouse 208, moves the mouse 208, or moves a scroll on the mouse 208. Additionally, the processor may create a reference model based on patient-observer interaction data that was stored in the database. Further, the database may include various algorithms for generating an alertness value, an optimal value for alertness, an interaction rate, a preferred interaction rate, or a threshold value (further described herein). The processor may execute an algorithm for performing a statistical analysis or various operations that result in generation of the alertness value, the optimal value for alertness, the interaction rate, the preferred interaction rate, or the threshold value.

The primary monitor 204 may be used by the patient-observer to monitor one or more patients in various hospital rooms. The primary monitor 204 may comprise an imaging sensor or camera directed at a face of the patient-observer. A pattern recognition algorithm and an image analysis may be used to identify the patient-observer's face from other objects in a room, identify eyes on the face, and send information to a processor to determine eye metrics. The imaging sensor and a digital signal processing circuitry may provide information to the processor as well. Performance of the pattern recognition algorithm and the image analysis may be improved or made user specific, for example, by using the camera or the imaging sensor to take reference images of the face with the eyes open, closed, and partially closed. The reference images may serve as standards for a pattern matching algorithm.

The user interface 206 may be a tablet for a user to interact with, another monitor, a smartphone, another monitor comprising a touchscreen, etc. The user interface 206 may be mounted to a table or may be unmounted. The user interface 206 may comprise a fingerprint sensor, a touch-sensitive display, a camera, buttons, audio components, etc. The user interface 206 may further comprise a sensor for detecting blood oxygen levels, stress, heart rate, and hydration. Sensors of the user interface 206 may be used in conjunction with other components of the exemplary central monitoring system 200 to record patient-observer interaction data. If one sensor is more accurate in a certain circumstance or for a particular type of data, data from that sensor may be used instead of another sensor.

The user interface 206 may also comprise a drawing tool 214 for use on the touch-sensitive display. The drawing tool 214 may detect patient-observer interaction data or inactivity in various ways, including but not limited to a sensor detecting a push of a button on the drawing tool 214, a touch sensor (e.g. detecting heat or pressure), a sensor detecting motion of the drawing tool 214, a sensor detecting a degree of speed of the motion of the drawing tool 214 (e.g. an acceleration sensor), a sensor detecting an orientation of the drawing tool 214, a sensor detecting pressure at the pen-point, etc. A sensor at the pen-point may detect when the drawing tool 214 comes within a proximity of the touchscreen (e.g. within 1 μm of the touchscreen or when the pen-point comes into physical contact with the touchscreen). The touch sensor may comprise a capacitive system or a light blocking system.

The mouse 208 may have various constructions. The mouse 208 may include single or a plurality of mechanical actuators (e.g. a left mechanical button and a right mechanical button) and the scroll. A surface of the mouse 208 may include one or more sensors that may allow for greater precision and accuracy for detecting touch or pressure depending on number, size, location, power supply, etc. of the one or more sensors. Information collected by the mouse 208 about the patient-observer interaction data or inactivity may be communicated to the remote computer 202 and stored in the database. Additionally, information corresponding to tracking a cursor of the mouse 208 may be stored in the database.

In one aspect, the mouse 208 may connect to the remote computer 202 via a wire or the mouse 208 may be wireless. The mouse 208 may be a wireless optical mouse that emits light (e.g. a laser, a light emitting diode configured to emit light toward a tracking surface) to detect movement of the wireless optical mouse. The mouse 208 may comprise power saving features, such as using a lower accuracy movement detection after a certain period of inactivity, wherein the lower accuracy movement detection consumes less power compared to when the mouse 208 detects movement. Further, the mouse 208 may selectively power down movement detection after a certain period of inactivity and selectively power up movement detection after the mouse 208 detects movement.

In another aspect, the mouse 208 may comprise a ball assembly including a ball and a sensor that detects movement of the mouse 208 based upon movement of the ball. The ball assembly may include a roller that rotates about an axis and is rotationally coupled to the ball. The ball assembly may include an additional roller that rotates about an axis and is rotationally coupled to the ball. The mouse 208 may comprise a disk including a magnet. The mouse 208 may comprise a Hall effect sensor and a current sensor.

The mouse 208 may detect patient-observer interaction data or inactivity in various ways, including but not limited to rotation of the roller about the axis, reflection of the light emitted from the mouse, a single multi-touch capacitive sensor, a resistive touch sensor, detection of heat from the patient-observer's hand, detection of pressure from the patient-observer's hand, detection of a click, detection of use of the scroll, etc. Touch sensors on the mouse 208 may cover a portion of a surface of the mouse 208 (e.g. 25% involving a left corner or 20% involving a top left corner and a bottom right corner), the total surface of the mouse 208, or substantially the entire surface of the mouse 208 (e.g. 75% of the mouse 208, excluding the right portion of the surface). Touch sensor may detect a touch of one or more fingers.

The keyboard 210 may comprise keys corresponding to characters for writing in a particular language, the keys comprising pressure sensors to detect when the patient-observer presses a particular key. The keyboard 210 may also comprise touch sensors, or heat sensors on the keys, wherein the sensors detect a near-keystroke (e.g. placement of a finger on a key without pressing down). The keyboard 210 may communicate received or detected data to the remote computer 202, which may be stored in the database. Received or detected data may include strokes of keys on the keyboard 210, use of a cursor button, or use of a touchpad.

The headset 212 may comprise a base that may be adjustable, the base having left and right speakers in earcups having a cushion. The headset 212 may comprise one or more earbuds having a speaker, connected wireles sly or by wire. The headset 212 may comprise a vocal activity detector connected to an acoustic sensor (e.g. one or more microphones). The vocal activity detector may detect when a patient-observer is speaking or taking heavy breaths. The vocal activity detector may detect changes in a patient-observer's voice, such as slower pronunciation for example. The acoustic sensor of the headset 212 may additionally comprise skin vibration sensors or electro-magnetic Doppler radar sensors for detecting vocal activity.

The headset 212 may comprise other contact sensors to detect electroencephalography data (“EEG”), electrooculography data, electromyography data, and electrocardiography data of the patient-observer. The other contact sensors may comprise a plurality of neural sensors on the base of the headset 212. The plurality of neural sensors may detect alpha, beta, gamma, and delta waves. Data from the headset 212 may detect deviations in brain activity (e.g. voltage, neural activity). EEG data may include measurements of neuro-signal voltage fluctuation from ionic current flows within brain neurons. EEG data may include brain electrical activity over a half hour interval. The headset 212 may also comprise a bone conduction sensor that touches the skin area of the user for detection of vibrations and bone conduction. The headset 212 may detect facial expressions and motor functions.

Various sensors in the headset 212 may employ various chemical, electrical, and/or optical technology to detect measurement of various environmental conditions as well as patient-observer characteristics and/or movements. Furthermore, sensor placement may be adjustable. Sensors in the headset 212 may be calibrated for control inputs based on one or more environmental operating conditions, such as ambient noise level or signal-to-noise ratios measured. The headset 212 may be any suitable electronic device configured to perform various functions and communicate information to various devices such as a computing device, a cell phone, a personal digital assistant, a gaming device, etc. Further, the headset 212 may communicate information wirelessly (e.g. through Bluetooth).

In addition, the processor may acquire audio data of multiple different patients for transmission to the headset 212. The processor may process the audio data by encoding the audio data with a compression function and communication network that has bandwidth sufficient to communicate the processed audio data to the headset 212. The processor may acquire the audio data from multiple different microphones in patient rooms associated with multiple different patients. The audio data from the patient rooms may be reproduced via a Web browser application to the patient-observer.

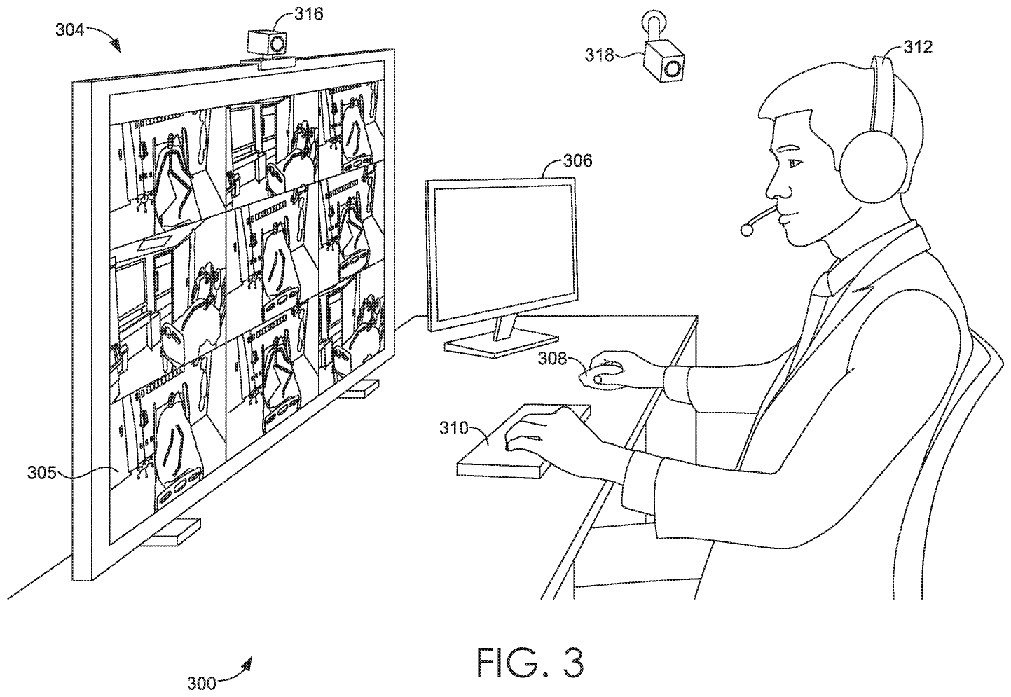

Turning to FIG. 3, an exemplary observation environment 300 comprises a primary monitor 304, a user interface 306, a mouse 308, a keyboard 310, a headset 312, a first detection device 316, and a second detection device 318. Exemplary observation environment 300 may additionally comprise, for example, a secondary monitor, additional user interfaces, a pager, and a cell phone. Exemplary observation environment 300 may additionally be configured as a standing desk so that the patient-observer may stand during observation. The primary monitor 304 may comprise a gyroscope to determine an angle of the primary monitor 304 relative to a desk. The patient-observer may use the primary monitor 304 to monitor various patients in the same hospital or in different hospitals. For example, monitoring area 305 provides for observation of a patient using a stick model. Other monitoring areas may provide for observations of a patient using a blob model (not depicted).

The first detection device 316 may be a camera attachable to the primary monitor 304, may be part of the primary monitor 304 (not depicted), or may be a cellphone-based camera. The first detection device 316 may include an image sensor and an optical component (e.g., camera lens). The first detection device 316 may be a digital camera configured to measure facial expressions, body posture, and head posture. The first detection device 316 may be configured to recognize the face of the patient-observer using the exemplary observation environment 300. The first detection device 316 may be configured to measure slouching and head tilt. The first detection device 316 may also be configured to measure eye metrics including, but not limited to, blinking rate, retina movement, blinking frequency, and relaxed eyelid. For example, the first detection device 316 may detect changes in the blinking rate. Additionally, the first detection device 316 may detect whether the retina movement involves scanning the primary monitor 304 or fixating in one area. The measured eye metrics may indicate whether a patient-observer is lacking a particular alertness.

In an embodiment, the first detection device 316 comprises optical imagers and light sources for detecting eye movement, including, but not limited to, blinking. The light sources may emit light (e.g. visible light or near IR light) from the eye (e.g. retina) of the patient-observer. The emitted light may produce a measurable retroreflection that may be used for determining an interaction rate for a particular patient-observer. The first detection device 316 may also include reference light sources for emitting light causing a reflection from a cornea of a patient-observer's eye that is measurable. The measurable reflection may be used for determining the interaction rate for the particular patient-observer.

The first detection device 316 may be configured to determine pupil size and/or distance between the patient-observer and the first detection device 316 via a 3D depth sensor, an infrared sensor, a laser device, and/or a sonar device. The first detection device 316 may be configured to determine the height of the patient-observer relative to the primary monitor 304. The primary monitor 304 may comprise a gyroscope that may be in communication with the first detection device 316 such that the processor may determine the angle of the primary monitor device relative to the patient-observer. Data received from the first detection device 316 may be calibrated to a particular patient-observer. For example, exemplary observation environment 300 may adjust an optimal value for alertness after accounting for a particular patient-observer's unique pupil shape, quality of vision, etc.

The second detection device 318 may be a digital camera configured to measure body posture (e.g. slouching) and head posture. For example, the digital camera may detect slouching and may be calibrated to a particular patient-observer based on, for example, height and weight. The second detection device 318 may be configured to detect the patient-observer turning his or her head toward the left or right away from the primary monitor 304. The second detection device 318 may be configured to detect the patient-observer using a personal cell phone, which results in reduced attention or alertness with respect to the primary monitor 304. The second detection device 318 may utilize image recognition software or other data analysis software. Body posture, head posture, and changed body posture or head posture may be detected, for example, by identifying a first body posture and a first head posture and later identifying a second body posture and a second head posture.

The second detection device 318 may be a 3D motion sensor. A 3D motion sensor is an electronic device that contains one or more cameras capable of identifying individual objects, people and motion. The 3D motion sensor may further contain one or more microphones to detect audio. The one or more cameras can utilize technologies including but not limited to color RGB, CMOS sensors, lasers, infrared projectors and RF-modulated light. The 3D motion sensor may have one or more integrated microprocessors and/or image sensors to detect and process information both transmitted from and received by the various cameras. Exemplary 3D motion sensors include the Microsoft® Kinect® Camera, the Sony® PlayStation® Camera, and the Intel® RealSense™ Camera, each of which happens to include microphones, although sound capture is not essential to the practice of the disclosure.

The second detection device 318 may operate continuously, or intermittently (for example, running for a fixed period at defined intervals), or on a trigger (e.g., when a motion detector or light sensor is activated, suggesting activity in the room). The second detection device 318 may operate continuously at all times while the monitoring is occurring, regardless of whether the patient-observer is moving or not. The second detection device 318 may view the entire body of the patient-observer by placement in a manner sufficient for the patient-observer to be visible to the camera. Alternately, the second detection device 318 may view any portion of the patient-observer. The second detection device 318 may record video, or may forward video to the remote computer 202 or directly to a database for storage. Video is a series of sequential, individual picture frames (e.g., 30 frames per second of video). Video data may include 3D depth data, data defining one or more bounding boxes, skeletal object tracking data and/or blob or object tracking data. In some implementations, it may be desirable for the sensors to capture video only, or sound only, or video and sound.

Alternatively, or additionally, if a patient-observer is monitoring detailed images or video streams of patients on the primary monitor 304, the second detection device 318 may blur, pixelate, or otherwise obscure (e.g. automatically convert details of patients to cartoons, blocks, blobs, stick figures) images or videos captured from the primary monitor 304 or the user interface 306. This may be done to protect patient privacy and modesty. The second detection device 318 may collect and transmit data sufficient for measuring and analyzing a patient-observer, but transmit only sufficient image data for a partially obscured video. In other aspects, the second detection device 318 may be associated with a microprocessor for processing image and/or video data to make any images and/or videos of patients captured on the primary monitor 304 or the user interface 306 more difficult to distinctly identify. In some aspects, only 3D depth data, bounding box data, skeletal object tracking data and/or blob or object tracking data is transmitted, without video or still images.

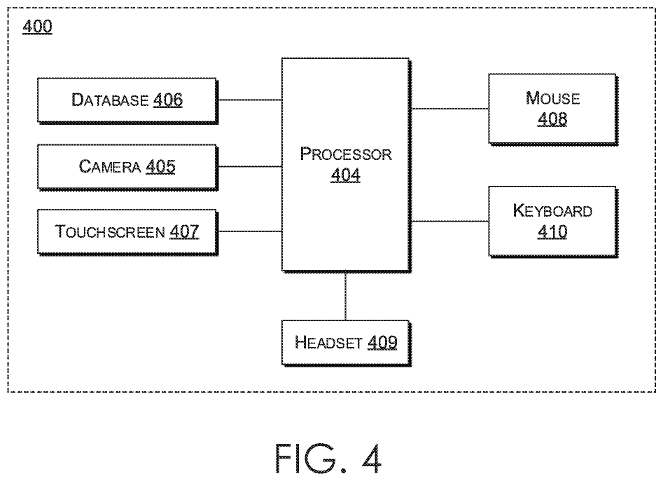

Turning to FIG. 4, exemplary system 400 for monitoring a patient-observer is a computing system comprising various devices and sensors. The exemplary system 400 comprises a processor 404, a camera 405, a database 406, a touchscreen 407, a mouse 408, and a keyboard 410. In an embodiment, exemplary system 400 includes one or more software agents. The one or more software agents may be implemented across a distributed cloud-computing platform. In some embodiments, the one or more software agents may be autonomous or semi-autonomous, adaptive, and capable of machine-learning.

In another embodiment, exemplary system 400 is an adaptive multi-agent operating system. The adaptive multi-agent operating system may employ decision making for applications such as, for example, searching, logical inference, pattern matching, and decomposition. The adaptive multi-agent operating system may provide capability to design and implement complex applications using formal modeling to solve complex problems. The adaptive multi-agent operating system may communicate via declarative messaging and use abstractions to allow for future adaptations and flexibility. An agent of the adaptive multi-agent operating system may have its own thread of control (e.g. for autonomy).

In yet another embodiment, exemplary system 400 may also take the form of an adaptive single agent system or a non-agent system. Further, exemplary system 400 may also be a distributed computing system, a data processing system, or a central monitoring system. Exemplary system 400 may comprise a single computer such as a desktop or laptop computer or a networked computing system. Exemplary system 400 may be configured to create a reference model; determine an optimal value for alertness using the processor 404, detect interaction data using the camera 405, the touchscreen 407, the mouse 408, the headset 409, and/or the keyboard 410; calculate alertness values using the processor 404; and generate warnings.

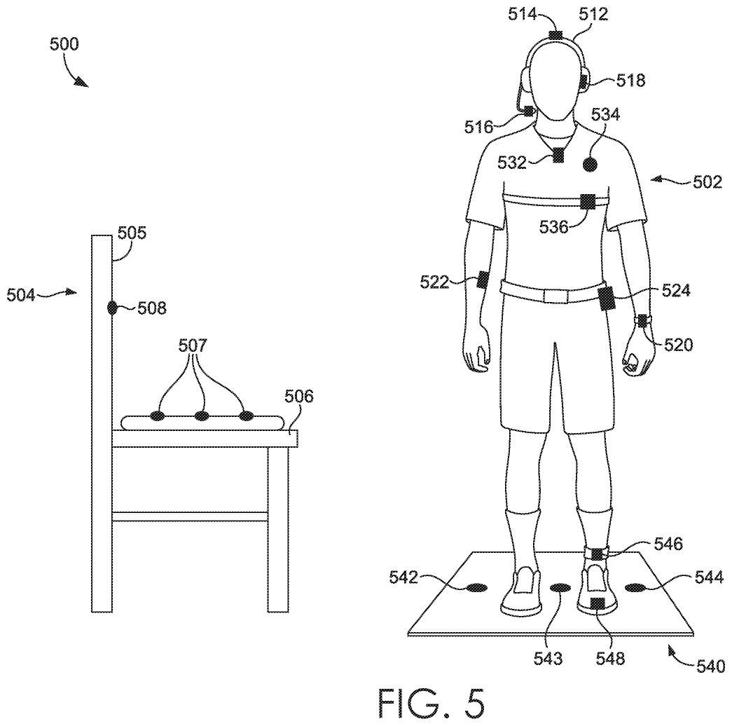

Turning to FIG. 5, an exemplary sensory environment 500 for the patient-observer is illustrated. Exemplary sensory environment 500 depicts a patient-observer 502, a seating unit 504, and a floor mat 540. The seating unit 504 may be a desk chair, a stool, a bench, a recliner, a rocker recliner, a glider, etc. (this is not an exhaustive list). The seating unit 504 may have a seating portion 506 comprising sensors 507. The sensors 507 may be on the seating portion 506 (not depicted) or on a cushion on top of the seating portion 506. Further, the seating unit 504 may have a back 505, wherein the back has a sensor 508. The back 505 and the seating portion 506 may have one sensor each or multiple sensors thereon.

Various types of sensors may be located on the seating unit 504. The various sensors may detect weight, pressure, and temperature, for example. These sensors may detect the weight of a patient-observer sitting on the seating unit 504 and various weight shifts made as the patient-observer is seated. For example, the patient-observer may be leaning forward and the sensors 507 may detect more weight or pressure at the front of the seating portion 506 away from the back 505. In an embodiment, sensors 507 and sensor 508 may comprise a pressure sensor on a base plate. Sensors 507 and sensor 508 may comprise a circuit board having electrodes. The circuit board may be located on the base plate. A membrane may be arranged so as to deflect under pressure and additionally establish electrical contact between electrodes.

Patient-observer 502 may wear a headset 512 comprising contact sensor 514, vocal sensor 516, and earphone 518. Earphone 518 may have speaker elements for listening to audio received in rooms of patients that the patient-observer is monitoring. The audio from a room of a patient is presented to the patient-observer and audio information from the room may be stored in a database and made accessible for later review using a computing device. The headset 512 may receive audio via a Web-enabled plug-in software component that allows the patient-observer to view a specific patient as part of the normal patient care management process. Audio data may be acquired from microphones and cameras comprising audio sensors located within patient rooms and is associated with specific patients via virtual electronic linkages that permit associating patient demographic information (including standard patient identifiers) with a patient location.

Contact sensor 514 may detect temperature, perspiration, or brain activity. Contact sensor may comprise a gyroscope or pressure sensor. In some embodiments, the contact sensor 514 includes a dry electrode for sensing neural signals when it is in contact with the scalp of the patient-observer. In some embodiments, the contact sensor 514 is integrated into an adjustable base of the headset 512. In some embodiments, the contact sensor 514 is on the exterior of the adjustable base of the headset 512. In some embodiments, there are multiple contact sensors in the adjustable base of the headset 512. In some embodiments, the contact sensor 514 is integrated into a cover or a pad attached to the adjustable base of the headset 512.

Head posture, temperature, perspiration, and brain activity data may be stored in a database over a period of time. Head posture, temperature, perspiration, and brain activity data from various patient-observers may be used for the creation of a reference model. Head posture, temperature, perspiration, and brain activity data may be used to determine an alertness value. The alertness value may be compared to the reference model. The reference model may be updated from time to time using new data gathered about head posture, temperature, perspiration, and brain activity.

Vocal sensor 516 may comprise pressure sensors for the detection of breathing. Vocal sensor 516 may detect a breathing rate and may further detect changes in the breathing rate. Since people usually breathe once every five seconds, vocal sensor 516 may detect whether shorter breaths or longer breaths are taken (e.g. three-second breaths or nine-second breaths). Vocal sensors may be calibrated to the particular patient-observer. For example, breathing rates could be adjusted for gender, weight, height, etc., to adjust for a proper calculation of an alertness value. Additionally, vocal sensors may detect slurring in speech or changes in speech volume from the beginning to the end of a shift, for example. Vocal sensors may detect changes in speech volume or the time it takes to pronounce a word, compared to previous shifts wherein the patient-observer spoke.

Patient-observer 502 may wear a watch sensor 520, a skin sensor 522, a waist strap sensor 524, a neck sensor 532, a badge sensor 534, and a chest strap sensor 536. Chest strap sensor 536 may be an external heart rate monitor in the form of an EKG sensor on a chest strap used to obtain heart rate data. The sensors worn by the patient-observer may obtain electrical signals associated with nerves, heart, or muscles of the patient-observer (e.g. electrical cardiac signals). The sensors worn by the patient-observer may comprise optical transducers for measuring chemicals in the skin. The optical transducers may detect carbon dioxide levels, oxygen levels, or a combination of these levels. Other examples of sensors the patient-observer may wear include a skin resistance sensor, an atmosphere pressure sensor, accelerometers, gyroscopes, temperature sensors (e.g. thermocouple, IR sensor), blood pressure sensors (e.g. a cuff), force transducers, conductance sensors, and respiration sensors.

A processor may control the frequency of measurements of a sensor. For example, an output of the transducer may be read ten times each second. The reading of a sensor may account for noise or other interferences to normalize data from the reading. Additionally, the processor may be in communication with a smartwatch that the patient-observer regularly wears, wherein the smartwatch comprises sensors that take various readings including, but not limited to, the daily exercise activity of the patient-observer, the sleep schedule of the patient-observer, the heart rate of the patient-observer, the weight of the patient-observer, the beverage intake of the patient-observer, the diet of the patient-observer, the blood pressure of the patient-observer, the blood glucose of the patient-observer, the caffeine intake of the patient-observer, and the level of hydration of the patient-observer. This data may be incorporated into the calculation of an alertness value for a particular patient-observer.

Exemplary sensory environment 500 may additionally include a floor mat 540 comprising sensors 542, 543, and 544. In some embodiments, the sensors 542, 543, and 544 are disposed on the upper surface of the floor mat 540 for detecting the position of the patient-observer's feet on the upper surface of the floor mat 540. In some embodiments, the sensors 542, 543, and 544 are integrated into the upper surface of the floor mat 540. For example, the sensors 542, 543, and 544 may be provided directly above or below the upper surface of the floor mat 540.

The sensors 542, 543, and 544 may include a force sensor that outputs data indicative of the patient-observer's foot positioning, such as whether more pressure is applied to the heel or toward the toes of a foot, whether only one foot is placed on the floor mat 540, or whether the feet are parallel to the patient-observer's hips. The sensors 542, 543, and 544 may also detect how far away the feet of the patient-observer are extended from the edge of the floor mat 540 closest to the patient-observer. The sensors 542, 543, and 544 may provide data together with data from the sensors 507 and the sensor 508 on the seating unit 504 for determining the positioning of the patient-observer's feet relative to the torso of the patient-observer. For example, sensor 508 may indicate the patient-observer's back is touching the back 505 of the seating unit 504. Continuing the example, the sensors 542, 543, and 544 may indicate right foot of the patient-observer is extended one inch from the edge of the floor mat 540 away from the patient-observer and away from the seating unit 504, such that the right foot is not pointing forward. This data may be used to calculate an alertness value for the patient-observer.

Patient-observer 502 may wear an ankle sensor 546 and a shoe sensor 548. The ankle sensor 546 and the shoe sensor 548 may comprise an accelerometer for detecting whether the patient-observer's foot is shaking and the rate at which it is shaking. The ankle sensor 546 may be attached around a sock or directly around the skin of the patient-observer. The ankle sensor 546 may be touching the skin of the patient-observer. The shoe sensor 548 may be attached to the outside of the shoe or inside the shoe (e.g. below the bottom of the sole of the foot under the heel or under the big toe, below the tongue of the shoe and above the top of the foot, and so forth). The ankle sensor 546 and the shoe sensor 548 may comprise haptic peripherals, such as a vibration, to warn a patient-observer if the patient-observer is inactive with a central monitoring system for a certain amount of time (e.g. to wake up a napping patient-observer).

In some embodiments, sensors may be worn in contact with the patient-observer, worn on the patient-observer's clothes, or elsewhere in the patient-observer's environment, depending on specific type of information that a sensor is intended to measure. In one embodiment, sensors include one or more accelerometers, gyroscopic meters, or combination of such devices as to enable one or more sensors to detect the patient-observer's motion, position or orientation, and changes in posture (e.g. slouching when the patient-observer has not slouched in the past four hours). Additionally, sensors may capture irregular periods of time between switching crossed legs or between switching from a crossed leg position to having both feet flat to the floor (e.g. switching crossed legs within thirty minute intervals to two minute intervals).

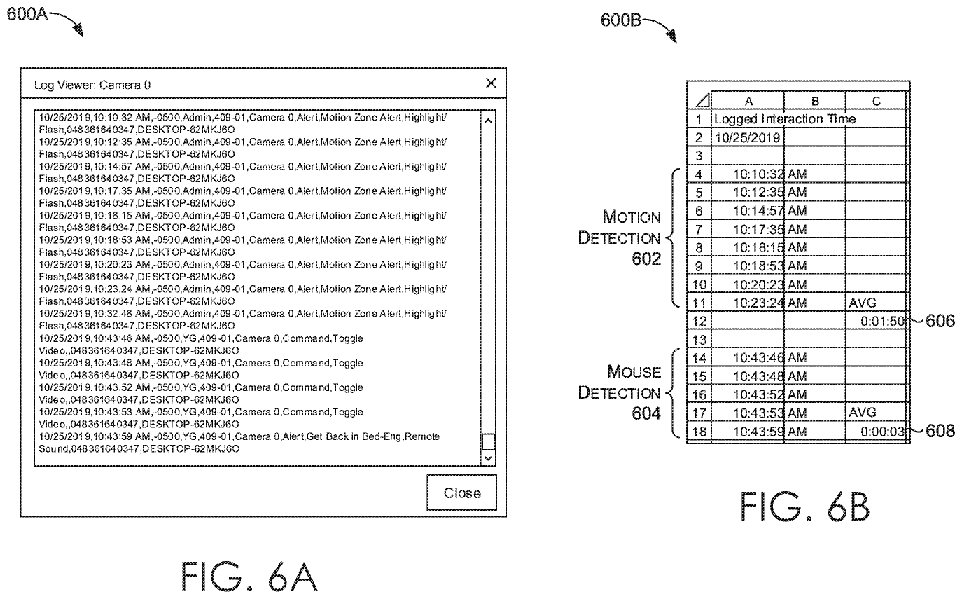

Turning to FIG. 6A, an exemplary log file 600A within a database comprises interaction data. Interaction data may be detected using the various sensors described above and stored in the database. For example, interaction data may comprise patient-observer interaction with exemplary central monitoring system 200 and data obtained from the first detection device 316 and the second detection device 318 in exemplary observation environment 300. As another example, interaction data may comprise data obtained by the sensors in exemplary sensory environment 500. For example, a camera on a monitor or tablet or a camera separate from a computing device may detect eye motion relative to a monitor wherein the patient-observer is monitoring videos of patients in real-time in various hospital rooms. As another example, the log file may store information as to each time a patient requires a certain action by the patient-observer and the action taken by the patient-observer. For example, a central monitoring system may indicate to the patient-observer that the patient needs to be asked a question. Continuing the example, the log file will record the time it took the patient-observer to ask the question through a microphone after the indication from the central monitoring system.

Exemplary log file 600A may record data of each time a patient-observer toggles between screens monitoring various patients. For example, the data may comprise the time each screen was toggled or the rate per minute at which each screen was toggled. Additionally, the exemplary log file 600A may record each time a patient-observer adjusts a camera (e.g. by using a computer to adjust the camera or pressing a button on a wireless device in communication with the camera). Further, the log file may record when a patient-observer has responded to a patient need. For example, a patient-observer may communicate through a microphone to a patient who has left his or her bed, the patient-observer may notify a nurse that the patient has left his or her bed, or the patient-observer may personally attend to the patient.

Interaction data may comprise each time the patient-observer used or touched a mouse, pressed or touched a key on the keyboard, and touched a touchscreen. Other interaction data includes how long the patient-observer stared at a particular screen on a monitor, how often the patient-observer was not looking at the monitor, or the breathing rate of the patient-observer. For example, if the patient-observer has a breathing rate above or below a certain threshold, the breathing rate may indicate the patient-observer is not optimally alert. Other interaction data may be stored in the log file, such as the amount of sleep of the patient-observer prior to a shift of monitoring patients and the amount of sugar consumed within the previous 24 hours before the shift. For example, sleep information and consumption of various foods or beverages may play a role in the level of alertness of a patient-observer during a particular shift. The sleep information could play a role in how a patient-observer interacts with a central monitoring system during the particular shift.

Turning to FIG. 6B, an exemplary logged interaction data 600B within the log file may comprise logged interaction times on a particular date, including detected motion data 602 and detected mouse data 604. The detected motion data 602 may comprise times of particular motions of the patient-observer and the detected mouse data 604 may comprise times of interaction with the mouse. Additionally, an average 606 of the detected motion and an average 608 of the detected mouse interaction may be obtained. The log file may also include predicted data based off the detected data, wherein data is predicted through various patterns, clusters, statistics, or combinations thereof.

The detected motion data 602 and the detected mouse data 604 may be used in various formulas, algorithms, and statistical models. Other detected data may comprise an amount of time a patient-observer looked at a particular screen on a monitor, which may be used in various calculations and flow charts. Additionally, other detected data may comprise the frequency or rate at which a patient-observer touches a keyboard or touchscreen. Interaction data may be recorded in a proprietary, industry standard, or open format. In general, all of the interaction data that may be detected may be included in the reference model.

The log file may be a starting point for when to generate a warning to a patient-observer when the patient-observer has an alertness value that does not satisfy an optimal value, when the patient-observer has an alertness value that fails to satisfy a threshold value, or when the patient-observer has an interaction rate that does not satisfy a preferred interaction rate. For example, data stored in the log file comprising interaction data of previous patient-observers may be used to initially generate an optimal value for alertness, a preferred interaction rate, or a threshold value for alertness. Interaction data from previous patient-observers may be imported from databases in various storage locations (e.g. in remote computers) or from a database cluster.

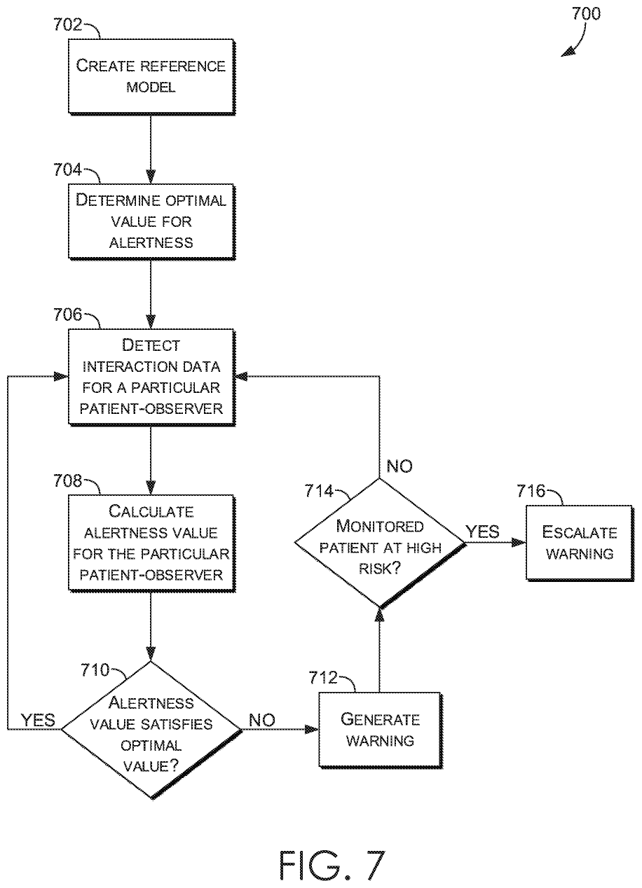

Turning to FIG. 7, exemplary flow diagram 700 provides for creating a reference model at step 702. The reference model may be based on interaction data of previous patient-observers. For example, interaction data may comprise patient-observer inactivity, such as failure to touch, move, click, or scroll the mouse; failure to speak into the headset; headset detection of a temperature range below that of a human head; or failure to touch a touchscreen. Further, the reference model may be updated in real-time using real-time interaction data as a patient-observer is monitoring one or more patients. In embodiments, average mouse clicks may be updated in the reference model after a shift of a particular patient-observer. The reference model may comprise information from a log file that is stored within a database. The reference model may be created by various algorithms, predictive models, statistical models, and combinations thereof. The reference model may be a graph or another type of trend indicator. The reference model may provide for a profile for each patient-observer comprising interaction data and demographic data (e.g. gender, height, weight, age).

Further, software management techniques may be utilized for the creation of the reference model. For example, logic could be applied to select which interaction data to utilize in the creation of the reference model and how much weight to give particular interaction data in the creation. Logic could also be applied to determine which interaction data are indicative for a particular type of alertness. For example, some interaction data may be more relevant for alertness relating to mental fatigue than for relating to excessive daytime sleepiness. The reference model may vary depending on the type of alertness and the type of interaction data. In some embodiments, there may be more than one reference model (e.g. one for each type of interaction data). Identification of which particular interaction data to use and how much weight to give particular interaction data may be based on trial-and-error, the most up-to-date analyses, or available operational data. The reference model may be trained using machine learning or other training (e.g. training a statistical model such as a Bayesian network).

At step 704, determining an optimal value for alertness may be based on the reference model. The optimal value for alertness may comprise an optimal value for an interaction rate with a mouse, a keyboard, or a touchscreen. For example, an optimal value for interaction rate with a mouse may be seven seconds per minute. This rate may include both clicking and mouse movement. As another example, an optimal value for interaction rate with both a mouse and a touchscreen may be nine seconds per minute (taking into account the lag between reaching to touch the touchscreen). The optimal value for alertness may comprise optimal values for patient-observer eye metrics including, but not limited to, blinking rate, rate of eye fixation, and blinking frequency.

Alternatively or additionally, the optimal value for alertness may vary depending upon the point in the shift. For example, the shift may permit a ten minute break after four hours. During the break, the optimal value may automatically adjust or it may adjust upon notification of a break by the patient-observer. As another example, the optimal value may differ at the start of the shift and after two hours into the shift. As another example, the optimal value may differ based on patient need and the hour of the day (e.g. midnight may have a lower optimal value because patients are sleeping, eight in the morning may have a higher optimal value because patients are waking up, and ten in the morning may have a lower optimal value when patients are being fed breakfast by hospital staff). The optimal value may also increase when a patient being monitored is having a physical reaction (e.g. getting out of bed or having a spasm) and the optimal value may decrease after fifty minutes of inactivity from the patients (e.g. the patient has fallen asleep and is not having a physical reaction).

At step 706, interaction data for a particular patient-observer may be detected. The interaction data may be detected using at least one sensor. For example, detection may occur through use of a camera capable of measuring facial expressions, head posture, slouching, etc. A first set of interaction data from a plurality of patient-observers may be detected, wherein the interaction data from a plurality of patient-observers comprises facial expressions, head posture, and slouching. A second set of interaction data from the plurality of patient-observers may be detected, wherein the second set of interaction data was detected by a mouse. A third set of interaction data from the plurality of patient-observers may be detected, wherein the third set of interaction data comprises eye metrics.

In some embodiments, the plurality of patient-observers includes previous patient-observers and a particular patient-observer who is currently working a shift. In some embodiments, the plurality of patient-observers includes a new group of patient-observers and some previous patient-observers. In some embodiments, the plurality of patient-observers includes only a new group of patient-observers (not including previous patient-observers). In some embodiments, the plurality of patient-observers includes only previous patient-observers.

At step 708, calculation of an alertness value for a particular patient-observer may involve data from the particular patient-observer stored in the cell phone of the particular patient-observer. For example, the exemplary central monitoring system 200 may import data from the particular patient-observer's cell phone comprising stress and/or relaxation levels through a combination of heart rate variability, skin conduction, noise pollution, and sleep quality. Continuing the example, calculating the alertness value may comprise a heart rate, wherein the calculating takes into account the gender, height, weight, age, and weekly average physical activity of the patient-observer.

Additionally, at step 708, calculation of an alertness value for a particular patient-observer may involve the rate at which the particular patient-observer touches the touch sensor on the mouse. For example, touch movement may be tracked for the particular patient-observer's finger on the sensor and may be compared to previous data. The comparison to previous data may provide for a calibration to insure that the particular patient-observer in fact touched the sensor. The comparison may also provide for corrected data to be used to update the alertness value. Further, information provided by the patient-observer may be used in the calculation of the alertness value. For example, the patient-observer may provide to a computing device the food consumed in the past twelve hours and the hours of sleep during the past forty eight hours.

The alertness value may comprise data from the plurality of neural sensors on the headset. A linear regression analysis may be performed to determine which interaction data detected by the plurality of neural sensors may provide more accurate alertness values. For example, different neural data may provide more accurate alertness information depending upon gender or depending upon whether detecting for a degree of arousal on a sleep-wake axis or a level of cognitive performance. In aspects, the alertness value may comprise interaction data from an IR sensor, wherein the data comprises a heart rate detected from a blood vessel, capillary, or vein. In other aspects, the alertness value may comprise interaction data from a sensor using light in the 500-600 nm range (the range in which blood absorbs light), wherein the interaction data includes a blood flow rate.

Additionally, the alertness value may comprise a variation of interaction data categories (e.g. mouse clicks, touches on screen), all interaction data detected, or a single set of interaction data involving only one category. For example, the alertness value may only involve interaction data detected by a mouse. Further, the alertness value may comprise interaction data detected for blinking rate, relaxed eyelid, and interaction data detected by a mouse and keyboard. The alertness value may comprise respiration rate determined from heart rate and blood flow rate.

At step 710, a determination is made as to whether an alertness value satisfies the optimal value. In an aspect, satisfaction comprises whether the alertness value is included within a range, whether the alertness value is excluded from the range, or whether the alertness value falls within a certain probability range of a normalized curve of interaction data detected and/or predicted. In another aspect, satisfaction comprises whether the alertness value is a certain percentage. In yet another aspect, satisfaction comprises whether various alertness values in combination reach a certain percentage or value. Satisfaction of the optimal value may include aggregation of alertness values over a period of time and analyzed for various interaction data categories (e.g. mouse clicks, touches on screen). Continuing the example, the aggregated alertness values may be analyzed based on the type of alertness.

If the optimal value is not satisfied, a warning is generated at step 712. If the optimal value is satisfied, then more interaction data may be detected (at step 706). At step 714, a determination is made as to whether one of the patients being monitored by the patient-observer is at a high risk. At step 716, if one of the patients is at a high risk, then the warning may be escalated. If not, then more interaction data may be detected (at step 706). Further, if more than one patient being monitored is at a high risk, then the warning may be even further escalated.

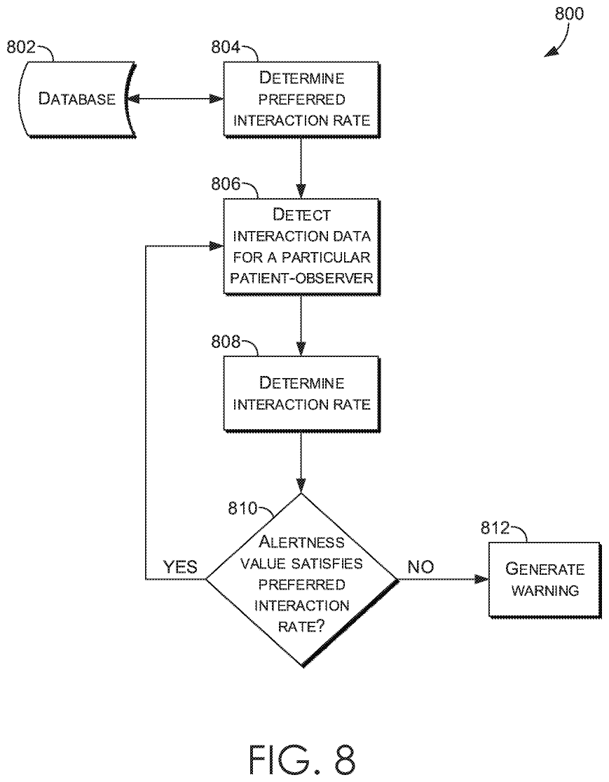

Turning to FIG. 8, exemplary flow diagram 800 provides for step 802 wherein a database maintains interaction data within a log file. At step 804, a preferred interaction rate for a particular patient-observer is determined based on interaction data of previous patient-observers, which is within the log file. The preferred interaction rate may be adjusted for probability of sensor error or sensor error rate, which are provided in a manufacturer specification or determined via testing. At step 806, interaction data for the particular patient-observer is detected. Interaction data may be detected using a sensor, including a mouse that may detect clicks, movement, and scrolling. Detected interaction data may be used to update the log file. At step 808, an interaction rate for the particular patient-observer is determined based on the detected interaction data from step 806. At step 810, a determination is made as to whether the interaction rate satisfies the preferred interaction rate. At step 812, if the preferred interaction rate is not satisfied, then a warning is generated. If the preferred interaction rate is satisfied, then the following step may return to step 806.

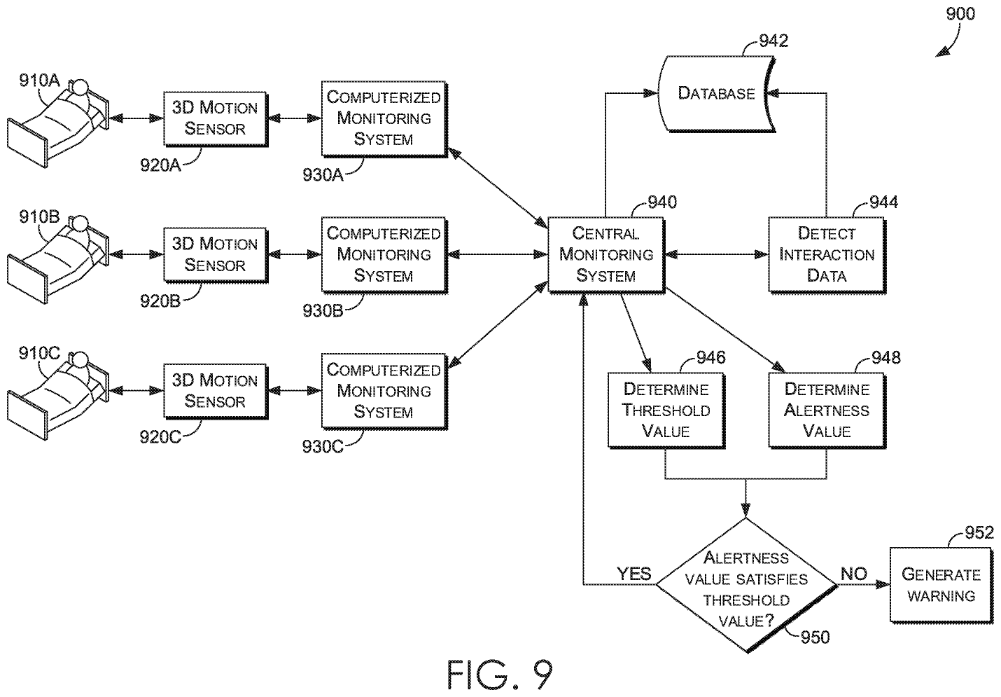

Turning to FIG. 9, exemplary flow diagram 900 provides for a system for monitoring a patient-observer as the patient-observer monitors patients. Patients 910A, 910B, and 910C are monitored by one or more 3D motion sensors 920A, 920B, and 920C, and the monitored data may be sent to computerized monitoring systems 930A, 930B, and 930C. The computerized monitoring systems 930A, 930B, and 930C monitoring systems may be associated one-to-one with each sensor or set of sensors 920A, 920B, and 920C. Information from the computerized monitoring systems 930A, 930B, and 930C is transmitted to a central monitoring system 940. However, in some aspects, data from set of sensors 920A, 920B, and 920C is transmitted directly to the central monitoring system 940. For example, the central monitoring system 940 may include hardware and software suitable for performing the tasks of the computerized monitoring systems 930A, 930B, and 930C. The central monitoring system 940 may comprise one or more processors, a camera, a monitor, and a mouse.

A patient-observer may monitor the patients 910A, 910B, and 910C through the central monitoring system. At step 942, a database may comprise interaction data within a log file. The log file may be used for creating a reference model. The central monitoring system 940 may be in communication with the database and the central monitoring system 940 may store data there. The interaction data in the log file may be from a plurality of patient-observers. Sensors may be in communication with the database, wherein the sensors may detect real-time interaction data of a particular patient-observer. At step 944, one or more sensors may detect real-time interaction data for the particular patient-observer. The central monitoring system 940 may be in communication with the one or more sensors. The detected interaction data may then be stored in the database at step 942. As detected interaction data is stored at step 942, the reference model may be updated using the detected real-time interaction data. At step 946, a threshold value for alertness may be determined using the one or more processors from the central monitoring system 940, wherein the one or more processors is in communication with the database and the one or more sensors. At step 948, an alertness value may be determined for a particular patient-observer by the one or more processors using the detected interaction data. At step 950, a determination is made as to whether the alertness value satisfies the threshold value. If the threshold value is not satisfied, then a warning is generated at step 952.

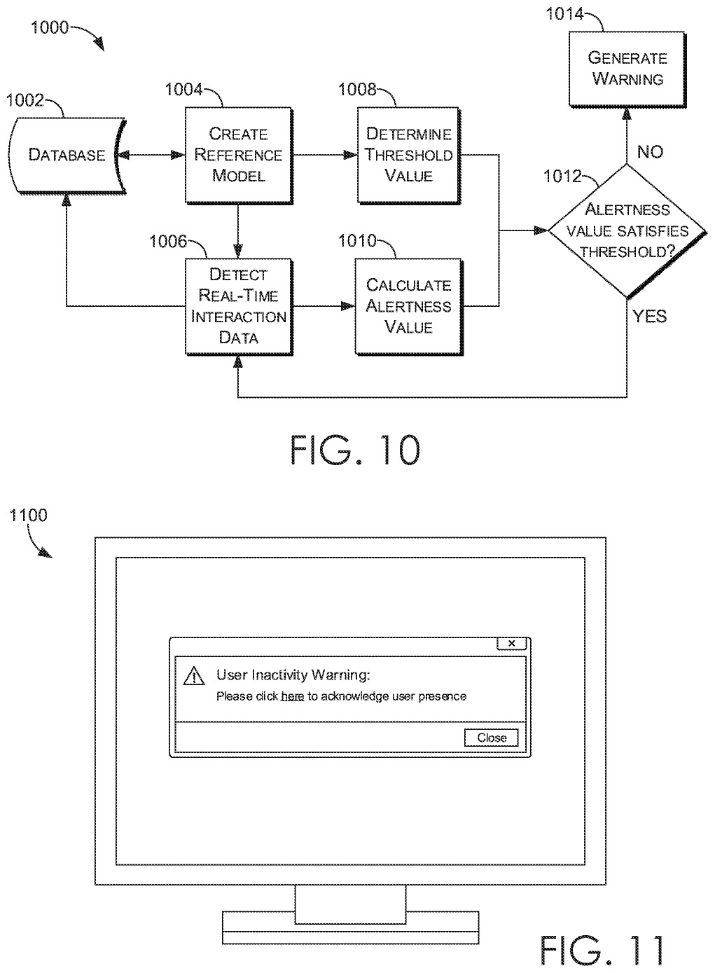

Turning to FIG. 10, exemplary flow diagram provides a database at step 1002, wherein the database comprises interaction data within a log file, and wherein the interaction data is from a plurality of patient-observers. At step 1004, a reference model may be created using one or more processors. In an aspect, because each patient-observer may experience a lack of alertness in different ways, the reference model may incorporate filtered or edited data based on a baseline model adjusted or filtered for demographic information. The baseline model may be an accumulation of all heart rate and breathing rate data for all patient-observers, which may then be adjusted or filtered by demographic information, such as gender, height, weight, age, ethnicity, and the like. Adjustments or filters may include one or more demographic information. Missing data or data not yet generated may be predicted using previous patient-observer interaction data. The missing data and the data not yet generated may be used in the reference model. In some embodiments, the missing data and the data not yet generated may be used to generate a predictive model. The predictive model may vary depending on the type of alertness and the type of interaction data.

At step 1006, real-time interaction data may be detected using one or more processors configured to utilize at least one sensor. The at least one sensor's margin of error may be accounted for and adjusted. The margin of error may be determined by testing or may be indicated by a manufacturer. As real-time interaction data is detected, the database at step 1002 may be updated with the real-time interaction data. With the updates to the database, the reference model may be updated (concurrently or subsequently) using the detected real-time interaction data. With the updates to the database, the predictive model may be updated (concurrently or subsequently) using the detected real-time interaction data.

At step 1008, a threshold value for alertness may be determined from the reference model. As the reference model updates, the threshold value may be updated (concurrently or subsequently). In some embodiments, the threshold value may be determined based on previous patient-observer interaction data only; based on previous patient-observer data and updated patient-observer interaction data; based on previous patient-observer data, updated patient-observer interaction data, and predicted interaction data; or updated patient-observer interaction data and predicted interaction data. Predicted interaction data may be based on missing data, data not yet generated, or both.

The threshold value may be represented as a percentage or unit of a normalized graph of the interaction data, wherein the threshold value falls within two standard deviations of the mean. In some embodiments, there is an upper threshold limit and a lower threshold limit. For example, a breathing rate or a heart rate may have an upper threshold limit and a lower threshold limit. The normalized graph may be based on a population of patient-observers or may be specific to the particular patient-observer, for example, by adjusting for demographic information. In some embodiments, adjustments and filters using one or more demographic information will provide for a more accurate determined threshold value with respect to the particular patient-observer. In some embodiments, the threshold value is automatically updated for particular patient-observer demographic information.

At step 1010, an alertness value for a particular patient-observer may be calculated based on detected real-time interaction data or a combination of real-time interaction data and previous interaction data of the particular patient-observer from prior shifts. At step 1012, a determination is made as to whether the alertness value satisfies the threshold value. In some embodiments, if the patient-observer's heart rate deviates from the baseline model or the adjustments or filters (as described in step 1004), the predictive model may determine whether the heart rate satisfies a threshold value of the reference model. In some embodiments, the patient-observer's alertness value may fail to satisfy the threshold value due to a medical emergency (e.g. heart attack, nose bleed, stroke, etc.). At step 1014, if the threshold value is not satisfied, a warning is generated.

Turning to FIG. 11, an exemplary alert or warning 1100 may be indicated to a patient-observer when the calculated alertness value does not satisfy the optimal value, when the interaction rate does not satisfy the preferred interaction rate, or when the alertness value fails to satisfy the threshold value of the reference model. An alert or warning may comprise a notification to a patient-observer on a monitor or other user interface. The notification may comprise a notice to the patient-observer to click a certain area to certify that the patient-observer is alert and ready to properly monitor the patients. If the patient-observer has a breathing rate that does not satisfy the threshold, the notification may comprise a prompt requesting a response from the patient-observer, wherein the response may indicate a reason for not satisfying the threshold or a request for assistance.

The alert or the warning may be escalated when a patient being monitored is at a high risk of falling off a monitored hospital bed, when a patient is at a high risk of having a seizure, when a patient is at a high risk of having a stroke, and so forth. For example, patients above a certain age and with certain conditions or symptoms may be at a high risk of falling off a monitored hospital bed. An escalated warning may include providing haptic peripherals (e.g. vibrations) to an ankle sensor on the patient-observer. Another escalated warning may include a notification to a supervisor, an alarm sound from a speaker within the room the patient-observer is monitoring patients, a signal to a remote site, other types of haptic feedback, and so forth.

Many different arrangements of the various components depicted, as well as components not shown, are possible without departing from the spirit and scope of the present invention. Embodiments of the present invention have been described with the intent to be illustrative rather than restrictive. Alternative embodiments will become apparent to those skilled in the art that do not depart from its scope. A skilled artisan may develop alternative means of implementing the aforementioned improvements without departing from the scope of the present invention.

It will be understood that certain features and subcombinations are of utility and may be employed without reference to other features and subcombinations and are contemplated within the scope of the claims. Not all steps listed in the various figures need be carried out in the specific order described. Accordingly, the scope of the invention is intended to be limited only by the following claims.

What is claimed is:

1. One or more non-transitory computer-readable media having executable instructions embodied thereon that, when executed by a processor of a computer device, perform a method, the method comprising:
creating a reference model based on interaction data of previous patient-observers;
determining an optimal value for alertness based on the reference model;
detecting, using at least one sensor, interaction data for a particular patient-observer;
calculating an alertness value for the particular patient-observer based on the reference model and the interaction data for the particular patient-observer; and
generating a warning when the calculated alertness value of the particular patient-observer does not satisfy the optimal value.

2. The media of claim 1, wherein the method further comprises:
detecting, using a camera capable of measuring facial expressions, head posture, and slouching, a first set of interaction data from a plurality of patient-observers, the first set of interaction data comprising facial expressions, head posture, and slouching;
detecting, using a mouse, a second set of interaction data from the plurality of patient-observers, the second set of interaction data comprising clicks, movement of the mouse, and scrolling; and
updating the reference model using the first and second sets of interaction data.

3. The media of claim 2, wherein the camera is further capable of detecting eye metrics including blinking rate, retina movement, blinking frequency, and relaxed eyelid.

4. The media of claim 3, wherein the method further comprises:
detecting, using the camera, interaction data from the particular patient-observer comprising facial expressions, head posture, slouching, blinking rate, retina movement, blinking frequency, and relaxed eyelid;
detecting, using the mouse, interaction data from the particular patient-observer comprising clicks, movement of the mouse, and scrolling; and
updating the alertness value using the detected interaction data from the particular patient-observer.

5. The media of claim 1, wherein the interaction data comprises a log file of each time a previous patient-observer interacted with a central monitoring system and an amount of time the previous patient-observer interacted with the central monitoring system.

6. The media of claim 5, wherein the interaction with the central monitoring system includes use of a mouse, a touchscreen, or a headset.

7. The media of claim 1, wherein the warning comprises a prompt to the particular patient-observer on a monitor.

8. The media of claim 1, wherein the interaction data comprises patient-observer inactivity with a central monitoring system.

9. The media of claim 1, wherein the optimal value is determined based at least partly on the at least one sensor's margin of error, and wherein the optimal value is automatically updated for the particular patient-observer based on demographic information.

10. The media of claim 1, wherein the interaction data comprises a heart rate, a respiration rate, and a blood pressure.

11. A computerized method for optimizing patient observation, the method comprising:
determining a preferred interaction rate for a particular patient-observer based on interaction data of previous patient-observers, the interaction data maintained in a log file within a database;
detecting interaction data for the particular patient-observer;
determining an interaction rate for the particular patient-observer based on the detected interaction data; and
generating a warning when the interaction rate does not satisfy the preferred interaction rate.

12. The method of claim 11, further comprising:
detecting, using a mouse, a set of interaction data from the previous patient-observers, the set of interaction data comprising clicks, mouse movement, and scrolling; and
updating the log file using the set of interaction data.

13. The method of claim 11, wherein the interaction data comprises information about each time a previous patient-observer interacted with a central monitoring system and an amount of time the previous patient-observer interacted with the central monitoring system.

14. The method of claim 13, wherein the interaction with the central monitoring system includes use of a mouse, a touchscreen, or a headset.

15. The method of claim 11, wherein the warning comprises a prompt to the particular patient-observer on a monitor.

16. The method of claim 11, wherein the interaction data for the particular patient-observer is detected using an electroencephalogram and additional sensors worn by the particular patient-observer.

17. The method of claim 11, wherein the preferred interaction rate is determined based at least partly on a sensor's margin of error, and wherein the preferred interaction rate is automatically updated for the particular patient-observer based on demographic information.

18. The method of claim 11, wherein the interaction data comprises a heart rate, a respiration rate, and a blood pressure.

19. A system for monitoring a patient-observer, the system comprising:
a database comprising interaction data within a log file, the interaction data from a plurality of patient-observers;
a sensor for detecting real-time interaction data of a particular patient-observer; and
one or more processors configured to:
create a reference model from the interaction data within the log file of the database;
detect, using the sensor, real-time interaction data for the particular patient-observer;
update the reference model using the detected real-time interaction data;
determine a threshold value for alertness from the reference model;
calculate an alertness value for the particular patient-observer; and
generate a warning when the alertness value for the particular patient-observer fails to satisfy the threshold value of the reference model.

20. The system of claim 19, wherein the warning is escalated when a patient being monitored is at high risk of falling off of a monitored hospital bed.
